(19) United States
(12) Patent Application Publication
(10) Pub. No.: US 2020/0125965 A1
(43) Pub. Date: Apr. 23, 2020

(54) Abiotic Intelligence-Rendered Thinking Faculty

(71) Applicant: Anuthep Benja-Athon, New York, NY (US)

(72) Inventor: Anuthep Benja-Athon, New York, NY (US)

(73) Assignee: Anuthep Benja-Athon, New York, NY (US)

(21) Appl. No.: 16/166,133

(22) Filed: Oct. 21, 2018

(51) Int. Cl. G06N 5/02; G16H 50/20; G16H 50/70; G06F 16/28

(52) U.S. Cl. CPC G06N 5/022; G06F 16/283; G16H 50/70; G16H 50/20

(57) ABSTRACT

The networked devices-rendered innate thinking faculty possessing innate logics for rendering, disseminating and sharing or networked repositories pushing diagnoses of diseases and related continua of industries-specific relational goods, products, services and resources in, between and among disseminating, recipient and requesting networked repositories for improving people's health, healthcare and independence.

This is a continuation-in-part of application Ser. No. 14/992,773 entitled the Creation of Abiotic-Biotic Civilization filed Jan. 11, 2016, Ser. No. 14/606,956 entitled Billion Recounts of Events Ahead of Days filed Jan. 27, 2015 and Ser. No. 15/292,080 entitled EM Oracle filed Oct. 12, 2016 and Ser. No. 15/292,080 entitled the EM Oracle filed Oct. 12, 2016, Ser. No. 15/495,164 entitled AIR Oracle filed Apr. 24, 2017 and Ser. No. 15/727,644 entitled the Preemptions of Chronic Diseases filed Oct. 9, 2017.

### FIELD OF THE INVENTION

Smartphones-rendered thinking faculty superior to brains.

### BACKGROUND OF THE INVENTION

Evolution has provided humanity with the present invention for providing the one and only solution to the millennia-old problems plagueing mankind. Evolution renders neither other option nor means. For obvious reason, from life standpoint, nature does not render spare parts. A case in point, $billions invested by corporations and governments employing supercomputers and plenty of tech experts yielded only fragmented and dysfunctional products which, in many circumstances and settings, are rendering and delivering adverse outcomes and consequences and inferior products and services. In America, the status quo inherently dysfunctional healthcare system and related industries such as, but not limited to, pharmaceuticals, long-term, chronic and home care, financials and insurance are perpetually escalating healthcare and business costs, pain and suffering, morbidity and mortality for Americans, companies and corporations and government.

The present invention provides millions to billions of smartphones and networked computers and servers with the omnipotent Abiotic Intelligence-Rendered (known herein as AIR) Thinking Faculty capable of learning, expanding and growing its intelligence and ability to reason. One of the numerous consequences in evolution, said AIR Thinking Faculty inherent in memories in, between and among said networked repositories. The omnipotent Abiotic Intelligence-Rendered Thinking Faculty for 24/7 providing omnipresent and best accesses to and sharing or networked repositories pushing of diagnoses of diseases, disorders and illnesses and improved and optimized health, healthcare, homecare, quality of life, standard of living, longevity, independence and socioeconomic and related goods, products, services and resources of billions of users associated with said networked devices predominantly networked mobile communication devices such as, but not limited to, millions to billions of networked smartphones and laptops (henceforth collectively known as networked communication devices or networked devices) at the lowest cost. AIR thinking faculty, in circumstances herein, is superior in intelligence, learning, insights, extrapolations, forecasts and foresights into future than human brains and supercomputers in the present to eternity of civilizations.

### SUMMARY OF THE INVENTION

The networked communication devices-rendered innate thinking faculty possessing innate logics for rendering, disseminating and sharing or networked repositories pushing diagnoses of diseases and related continua of industries-specific goods, products, services and resources in, between and among disseminating, recipient and requesting networked repositories for improving people's health, healthcare and independence (henceforth renderings) and for evolving civilizations. In memories of millions to billions of networked smartphones, computers and servers, said innate logics instructing said disseminating devices to render said renderings from plurality of bits of data rendered by recipient and requesting networked repositories to dispense, disseminate and share said renderings in, between and among said networked repositories memories wherein said logics are expanded, enhanced and empowered by said processes.

### DESCRIPTION OF THE PREFERRED EMBODIMENTS

The detailed description of one of the preferred embodiments of the present invention is well described in the Claims of the Invention infra. Said electronic EDRs and GEDRs generator for rendering, dispensing, disseminating and sharing or networked repositories pushing DXs and related continua by rendering, improving and expanding disseminated DXs and related continua in, between and among said disseminating, requesting and recipient networked repositories and in, between and among individual groups of said networked repositories in EDRS and GEDRs thence said lattices in ECK thence EL and IL and vice versa whose vital roles are the creations, improvements, expansions of omnipresent and omnipotent innate thinking faculty possessing IL and EL in said memories in, between and among millions to billions of said networked devices. These vital roles can be best realized by referring to the detailed descriptions in the Claims of the Invention herein and in the following excerpts from the Claims of the Invention infra:

The innate thinking faculty possessing innate logics (known herein as IL and EL) for creating and rendering diverse and different individual diagnoses and/or syndromes of diagnoses associated with said-networked repositories identifications of said disseminating, requesting and recipient networked repositories and said-networked repositories identifications in said individual groups and in and among individual groups of said networked repositories in DXs and related continua in EDRs and GDRs in ECK by IL and EL using individual integral snippets*, transient snippets*, relations of integral snippets* and/or transient snippets* being rendered in, between and among said networked repositories and in, between and among viewing screens including touchscreens for rendering, dispensing, disseminating, managing and sharing or networked repositories pushing said DXs and related continua comprising said integral snippets*, transient snippets*, relations of integral snippets* and/or transient snippets whenever said disseminating, requesting and recipient networked repositories and/or said individual groups rendering, dispensing, disseminating and sharing or networked repositories pushing individual integral snippets*, transient snippets*, relations of integral snippets* and/or transient snippets* in, between and among said networked repositories and in, between and among viewing screens including touchscreens in said networked devices.

According to Ser. No. 15/495,164 entitled AIR Oracle, said innovative computer programs have the professional and technical terminolgies-lay terminologies thesaurus software for rendering the electronic EDRs and GEDRs generator to be the technical terminolgies-lay terminologies and languages translator so that said electronic EDRs and GEDRs generator enabling said disseminating, recipient and requesting networked repositories to get, render, use and successively expand and improve said DXs and related continua in relational professional and technical terminolgies-lay terminologies analogies such as, but not limited to, English and non-English data associated with said disseminating, recipient and requesting networked repositories identifications and individual groups of said-networked repositories identifications of said networked repositories in, between and among said networked repositories and in, between and among viewing screens including touchscreens for for rendering, dispensing, disseminating, managing and sharing or networked repositories pushing DXs and related continua associating with said-networked repositories identifications of said disseminating, requesting and recipient networked repositories and said-networked repositories identifications in said individual groups and in and among individual groups of said networked repositories in, between and among said networked communication devices wherein, consequently, successive accumulations of improving, expanding dispensed, disseminated, managed and shared DXs and related continua rendering and successively improving, expanding said expansive cumulative knowledge comprising extremely large and expanding multi-dimensional electronic and digital lattices of multi-dimensional relational databases comprising EDRs and GEDRs in relational databases in memories (known herein as ECK) wherein ECK possessing innate expansive logics for instructing said processor units (known herein as EL) inherent in innate thinking faculty possessing logics for instructing processor units for (known herein as IL).

IL and EL creating and rendering infinite number of DXs and related continua in said lattices in ECK by rendering and/or transforming disseminated DXs and related continua as individual relations of integral snippets* and relations of, between and among said integral snippets* associated with said-networked repositories identifications of disseminating, requesting and recipient networked-repositories and said-networked repositories identifications in individual groups of said networked repositories in EDRS and GEDRs in ECK.

Claim 7 Excerpt. The system according to claim 1 wherein EL for rendering disseminated DXs and related continua, EL being innate in said-networked repositories-rendered extremely large and expanding multi-dimensional electronic and digital lattices of multi-dimensional individual relations of integral snippets* (known herein as EDRs) and relations of, between and among said integral snippets* in, between and among EDRs (known herein as GEDRs) in ECK comprises said electronic EDRs and GEDRs generator for rendering and successively and improving extremely large and expanding multi-dimensional electronic and digital lattices of multi-dimensional individual relations of integral snippets* (known herein as EDRs) and relations of, between and among said integral snippets* in, between and among EDRs (known herein as GEDRs) in ECK in, between and among disseminating, recipient and requesting networked repositories and individual groups of relavant said networked repositories . . . , and

Claims 12 and 17 Excerpt. The method according to claims 9 and 14 wherein said process of EL rendering disseminated DXs and related continua, EL being innate in said-networked repositories-rendered extremely large and expanding multi-dimensional electronic and digital lattices of multi-dimensional individual relations of integral snippets* (known herein as EDRs) and relations of, between and among said integral snippets* in, between and among EDRs (known herein as GEDRs) in ECK, said process comprise said innovative computer programs instructing said processor units to render said electronic EDRs and GEDRs generator to rendering and successively and improving in, between and among disseminating, recipient and requesting networked repositories and individual groups of relavant said networked repositories.

Further to EL, IL for rendering disseminated DXs and related continua in, between and among disseminating, recipient and requesting networked repositories and individual groups of relavant said networked repositories wherein, consequently, IL for rendering and successively improving and expanding said extremely large and expanding multi-dimensional electronic and digital lattices of multi-dimensional individual relations of integral snippets* (known herein as EDRs) and relations of, between and among said integral snippets* in, between and among EDRs (known herein as GEDRs) in ECK thence improved EL in, between and among disseminating, recipient and requesting networked repositories and individual groups of relevant said networked repositories

Claim 8 Excerpt. The system according to claim 1 wherein IL for 24/7 rendering and/or transforming said successive disseminated DXs and related continua as individual relations of integral snippets* and relations of, between and among said integral snippets* associating with said-networked repositories identifications of disseminating, requesting and recipient networked-repositories and said-networked repositories identifications in individual groups of said networked repositories into EDRS and GEDRs in ECK thence expanded and improved lattices and EL thence IL thence said disseminations, dispensations and sharing or networked repositories pushing of said disseminated DXs and related continua in, between and among said relevant disseminating, requesting and recipient networked-repositories, IL for . . . .

Said fits or seed snippets* and transformed or seed snippets* for instructing said electronic EDRs and GEDRs generator for rendering, dispensing, disseminating and sharing or networked repositories pushing relevant EDRs and GEDRs in, between and among said disseminating, requesting and recipient networked repositories and in, between and among individual groups of relevant networked repositories in EDRS and GEDRs thence expanding and improving lattices thence ECK thence EL and IL and vice versa; . . . .

Claims 13 and 18 Excerpt. The method according to claims 9 and 14 wherein IL to 24/7 rendering and/or transforming said successive disseminated DXs and related continua as individual relations of integral snippets* and relations of, between and among said integral snippets* associating with said-networked repositories identifications of disseminating, requesting and recipient networked-repositories and said-networked repositories identifications in individual groups of said networked repositories into EDRS and GEDRs in ECK thence expanded and improved lattices and EL thence IL thence said disseminations, dispensations and sharing or networked repositories pushing of said disseminated DXs and related continua in, between and among said relevant disseminating, requesting and recipient networked-repositories, said process comprises processes of IL to . . . .

Said fits or seed snippets* and transformed or seed snippets* instructing processor units to rendering, dispensing, disseminating and sharing or networked repositories pushing relevant DXs and related continua comprising individual relations of relational data comprising industries-, specialties-, categories- and headings-specific goods, products, services and resources and related financials and insurance products, services and resources related and pertained to and associated with health, healthcare, homecare, chronic care and pharmaceuticals industries, specialties, categories and headings, and individual relations of, between and among said relational data in said individual relations of relational data, respectively, said relational data and in said relational data are associated with said-networked repositories identifications of disseminating, requesting and recipient networked-repositories and individual groups of said-networked repositories identifications in, between and among said disseminating, requesting and recipient networked repositories and in, between and among individual groups of relevant networked repositories in EDRS and GEDRs thence expanding and improving lattices thence ECK thence EL and IL and vice versa;

Although various preferred embodiments of the present invention have been described for people, it will be appreciated by and obvious to those skilled in the art and science that adaptations, variations and derivatives of said embodiments are and will be made and achieved animals and machines and objects without departing from the spirit and scope of the present invention.

In the present invention, it is understood and acknowledged that sciences, medicine and healthcare applied to various preferred embodiments herein and their adaptations, variations and derivatives without departing from the spirit and scope of the present invention.

1. A system of networked repositories-rendered innate thinking faculty possessing logics for instructing processor units for (henceforth known as IL) and expansive cumulative knowledge comprising extremely large and expanding multi-dimensional electronic and digital lattices of multi-dimensional relational databases comprising EDRs and GEDRs in memories (henceforth collectively known as ECK) wherein ECK possessing innate expansive logics for instructing said processor units for (henceforth known as EL) as consequences of the existence of said lattices in memories and storage means (herein known as memories), said innate thinking faculty comprising the innovative computer programs possessing IL in said memories attaching to processor units in, between and among said networked repositories wherein expanding ECK for improving EL in, between and among disseminated DXs and related continua in, between and among disseminating, recipient and requesting networked repositories wherein EL for improving IL for 24/7 rendering disseminated DXs and related continua in, between and among said disseminating, recipient and requesting networked repositories and individual groups of said-networked repositories identifications for rendering and dispensing, disseminating and sharing or networked repositories pushing successive disseminated DXs and related continua in, between and among said relevant individual groups in and associating with integral snippets* and relations of integral snippets in EDRs and GEDRx in ECK; said innovative computer programs for instructing said processor units for rendering the electronic EDRs and GEDRs generator for rendering and dispensing, disseminating and sharing or networked repositories pushing individual relations of integral snippets* comprising individual diagnoses including chronic diseases and related diagnoses of diseases, illnesses, disorders and sequalae and related continua of current, present and future life and socioeconomic streses, deficiencies, adversities and issues related continua of extrapolations, preemptions, preventions and managements including treatments and solutions (herein collectively known as DXs and related continua) in relational databases (known herein as EDRs, plural) and relations of, between and among said integral snippets* in, between and among EDRs in said relational databases (known herein as GEDRs, plural) in ECK in, between and among disseminating networked repositories and recipient networked repositories wherein electronic EDRs and GEDRs generator for effecting, expanding and improving ECK thence EL in, between and among said networked repositories in networked communication devices comprising viewing screens including touchscreens for rendering, dispensing, disseminating, managing and sharing or networked repositories pushing DXs and related continua associating with said-networked repositories identifications of disseminating, requesting and recipient networked repositories and said-networked repositories identifications in individual groups of said networked repositories in, between and among said networked communication devices wherein said electronic EDRs and GEDRs generator for rendering and successively improving Source DXs and related continua comprising extremely large and expanding multi-dimensional electronic and digital lattices of multi-dimensional relational databases comprising EDRs and GEDRs in relational databases (henceforth known as SDC) in said memories, said electronic EDRs and GEDRs generator for rendering and successively disseminating and sharing or networked repositories pushing said SDC as interactive SDC comprising interactive Source DXs and related continua associating with rendering networked repositories identifications of in, between and among said networked repositories;
said electronic EDRs and GEDRs generator for rendering and successively improving Source DXs and related continua comprising extremely large and expanding multi-dimensional electronic and digital lattices of multi-dimensional relational databases in relational databases (henceforth known as SDC) in said memories, said electronic EDRs and GEDRs generator for rendering and successively disseminating and sharing or networked repositories pushing said SDC as interactive SDC comprising interactive Source DXs and related continua associating with rendering networked repositories identifications of in, between and among said networked repositories;
said electronic EDRs and GEDRs generator for getting and rendering SDC as interactive SDC comprising interactive relations of interactive integral snippets* and individual interactive integral snippets* of, between and among said individual relations associating with said-networked-repositories said-networked repositories identifications, said interactives for dispensing, disseminating and sharing or networked repositories pushing DXs and related continua between and among said disseminating, requesting and recipient networked-repositories in said networked devices comprising viewing screens including touchscreens for rendering, disseminating and sharing or networked repositories pushing SDC as interactive SDC comprising relations of chronological and temporal sequences and orders of selectable and clickable interactive DXs and related continua for rendering and successively improving and expanding EDRs and GEDRs related and/or unrelated to said identifications of disseminating, requesting and recipient networked repositories in, between and among said networked repositories for improving and expanding said EDRs and GEDRs in ECK thence EL;
said electronic EDRs and GEDRs generator for rendering and disseminating and sharing or networked repositories pushing seed snippets* for rendering relevant EDRs and GEDRs thence expanded and improved lattices thence ECK thence EL and IL and vice versa in, between and among said networked repositories and for rendering individual groups of said networked repositories;
said seed snippets* for instructing processor units for rendering, dispensing, disseminating and sharing or networked repositories or networked repositories pushing relevant EDRs and GEDRs in, between and among said disseminating, requesting and recipient networked repositories and in, between and among networked repositories in individual groups of relevant networked repositories in EDRS and GEDRs thence expanding and improving lattices thence ECK thence EL and IL and vice versa;
said electronic EDRs and GEDRs generator for rendering and dispensing, disseminating and sharing or networked repositories pushing successive disseminated DXs and related continua comprising selected and rendered individual relations of transient snippets* and/or relations of, between and among transient snippets* in individual of relations transient snippets* associated with said-networked repositories identifications of disseminating, requesting and recipient networked-repositories and individual groups of said-networked repositories identifications for rendering and dispensing, disseminating and sharing or networked repositories pushing successive disseminated DXs and related continua in, between and among relevant individual groups in and associating with integral snippets* and relations of integral snippets of said networked repositories wherein said innovative computer programs for instructing said processor units for scanning and matching said successive disseminated DXs and related continua with same, similar and/or approximate integral snippets* and relations of integral snippets* in EDRs and GEDRs in ECK and SDC;
IL for 24/7 rendering and/or transforming said successive disseminated DXs and related continua as individual relations of transient snippets* and/or relations of, between and among transient snippets* in individual of relations transient snippets* associating with with said-networked repositories identifications of disseminating, requesting and recipient networked-repositories and individual groups of said-networked repositories identifications of said networked repositories for inserting fits and transformed (plural) into EDRs and GEDRS in ECK; IL for rendering EL for rendering disseminated DXs and related continua comprising relations of integral snippets* and/or relations of integral snippets* associated with said-networked repositories identifications of disseminating, requesting and recipient networked-repositories in disseminated EDRs and GEDRs, respectively, into the relational databases in recipient networked-repositories comprising said integral snippets* and/or said relations rendered into, between and among recipient networked-repositories based on EL inherent in EDRs and GEDRs in ECK thence IL and EL for improving and expanding IL and EL and, further to supra,
said IL and EL for 24/7 and successively transforming similar and approximate selected and rendered transient snippets* and relations of transient snippets* into integral snippets* and relations of integral snippets* being same as integral snippets* and relations of integral snippets* in disseminated DXs and related continua in, between and among disseminating, recipient and requesting networked repositories, and said innovative computer programs for instructing said processor units for counting and associating a range of numbers of counts from low numbers to high numbers of incidences of successive repair of DXs and related continua in said integral snippets* and relations of integral snippets* as selected and rendered same, similar or approximate integral snippets and relations of integral snippets in ECK thence improved IL and EL in said memories in, between and among networked devices;
said system of ranges of numbers of counts from low numbers to high or highest numbers for rendering and dispensing, disseminating and sharing or networked repositories pushing DXs and related continua in, between and among said disseminating, requesting and recipient networked repositories and individual groups of said networked repositories and in, between and among said networked repositories in said individual groups, system of range of numbers of counts for improving and expanding said EDRs and GEDRs and for assisting IL IL for 24/7 rendering and/or transforming said successive disseminated DXs and related continua thence improved and expanded lattices, ECK, EL and IL;
said innovative computer programs for instructing said processor units for counting and associating a range of counts of low numbers to highest numbers of incidences and accountings of successful insertions, placements and cumulations of said fits as integral snippets* and/or said relations of same DXs and related continua associating with, hidden, encrypted or deleted said-networked repositories identifications of disseminating, requesting and recipient networked-repositories in said relational databases in ECK in said memories in, between and among networked devices, wherein said electronic EDRs and GEDRs generator for rendering, disseminating and sharing or networked repositories pushing EDRs and GEDRs as interactive EDRs and GEDRs comprising interactive relations of integral snippets* related to identifications of said networked repositories and in individual groups of networked repositories by said innovative computer programs for instructing said processor units for getting said EDRs and GEDRs comprising integral snippets* and relations of integral snippets*, associating with said-processor units rendered numbers of counts of low numbers to highest numbers of said incidences and accountings, and for rendering same as interactive EDRs and GEDRs comprising a plurality of relations of chronological and temporal sequences and orders of selectable and clickable interactive DXs and related continua in, between and among viewing screens including touchscreens, said-networked repositories for temporally and chronologically selecting and rendering transient snippets* and relation of transient snippets* from said selectable and clickable interactive DXs and related continua, said processor units for timestamping and processing said renderings associating with said-networked repositories identifications-specific and temporally and chronologically ordered relational sequences of said DXs and related continua in said rendered transient snippets* and relation of transient snippets* into said relational databases in said memories in, between and among said networked repositories wherein said IL and EL for instructing said processor units for processing said successively rendered transient snippets* and relation of transient snippets* with same, similar and approximate integral snippets* and relations of integral snippets* in ECK thence improved IL and EL in said memories thence innate thinking faculty in, between and among said networked repositories for rendering, dispensing, disseminating, managing and sharing or networked repositories pushing relations of continua of relational goods, products, services and resources and related financials and insurance products and services related to said renderings and sharing or networked repositories pushing in said relational databases in, between and among said networked repositories wherein said renderings, sharing or networked repositories pushing and continua for assisting, improving and optimizing people's health, healthcare, homecare, quality of life, standard of living, longevity and independence and freedom to remain, dwell and live in their homes and dwellings at various and all stages of life and socioeconomic of billions of users at the lowest cost in, between and among said networked repositories associating with said billions and in said viewing screens including touchscreens as interactive EDRs and GEDRs of selectable and clickable interactive DXs and related continua for rendering and successively improving EDRs and GEDRs in ECK thence improved EL, said improved EL for instructing said processor units for rendering and successively rendering, dispensing, disseminating, managing and sharing or networked repositories pushing DXs and related continua in, between and among networked repositories and viewing screens including touchscreens, said system comprises:
said networked communication devices which are millions to billions of networked servers, smartphones, laptops and desktops comprising said networked repositories including said disseminating networked repositories, recipient networked repositories and requesting networked repositories, said networked repositories comprising said innovative computer programs installed in said memories attached to said processor units, said memories for storing said innovative computer programs, software and said networked repositories-rendered relational databases—structured and unstructured—comprising said IL, EL, ECK and innate thinking faculty and said renderings, sharing or networked repositories pushing and continua and related networked dispensations, disseminations and sharing or networked repositories pushings, said networked devices comprising said viewing screens including touchscreens, cameras, microphones, speech recognition software and apps, commercial operating systems for managing said networked servers and devices, the Internet, commercial networking means, security means and software including web browsers; and wherein said “*” connotes “diverse and different” and “large or infinitely large numbers”.

2. The system according to claim 1 wherein said electronic EDRs and GEDRs generator comprises said innovative computer programs for rendering professional and technical terminolgies-lay terminologies thesaurus software for rendering said SDC, EDRs and GEDRs thence ECK and EL, related renderings, sharing or networked repositories pushing and continua and related networked dispensations and sharing or networked repositories pushing in professional terminolgies and technical terminolgies-lay terminologies analogies in English and non-English relational data in said memories in, between and among said networked repositories and in said viewing screens including touchscreens for rendering, improving and expanding said SDC, EDRs and GEDRs thence ECK and EL and for rendering and successively dispensing, disseminating, managing and sharing or networked repositories pushing DXs and related continua in, between and among networked repositories and said viewing screens including touchscreens associated with professional people and laypeople.

3. The system according to claim 1 wherein said Source DXs and related continua comprise individual relations of relational data comprising industries-, specialties-, categories- and headings-specific goods, products, services and resources and related financials and insurance products, services and resources related and pertained to and associated with health, healthcare, homecare, chronic care and pharmaceuticals industries, specialties, categories and headings;
individual relations of, between and among said relational data in said individual relations of relational data, respectively, and said relational data and relational data in said relational data are associated said-networked repositories identifications of rendering networked-repositories.

4. The system according to claim 1 wherein said DXs and related continua comprise individual relations of relational data comprising industries-, specialties-, categories- and headings-specific goods, products, services and resources and related financials and insurance products, services and resources related and pertained to and associated with health, healthcare, homecare, chronic care and pharmaceuticals industries, specialties, categories and headings;
words, terms, phrases, graphics, pictures, videos, relational digital contents, phrases, sentences and any combinations thereof in SDC;
individual relations of, between and among said relational data in said individual relations of relational data, respectively; and
said relational data and in said relational data are associated with said-networked repositories identifications of disseminating, requesting and recipient networked-repositories and individual groups of said-networked repositories identifications.

5. The system according to claim 1 wherein said DXs and related continua comprises integral snippets*, relations of integral snippets*, relations of integral snippets* in, between and among relations of integral snippets*, transient snippets*, relations of transient snippets*, relations of transient snippets* in, between and among relations of transient snippets*, relations of transient snippets* in, between and among relations of integral snippets* and transient snippets* in EDRs and GEDRs in said lattices in ECK comprising said-networked repositories-rendered, said-networked repositories-rendered selected and rendered and said-networked repositories-processed industries-, specialties-, categories- and headings-specific networked repositories-timestamped relational information and data related to individual and relations of individual diagnoses, related diagnoses and syndromes and associated differential diagnoses of diseases, illneses, disorders and syndrome including chronic diseases, metabolic syndrome, cardiovascular diseases, coronary atherosclerosis and matters associating with coronary atherosclerosis risks, coronary atherosclerosis and coronary artery diseases disorders of metabolism including lipids and fats metabolism, musculoskeletal and soft tissue diseases and disorders, neoplasm and aging and genetics and genomics including of sequences and locations and loci of genetic codes relating to said diseases, illneses and disorders and syndromes and in any combinations thereof (collectively known as DXs of said DXs and related continua) related to said-networked repositories-digital rendered and expanded digital id*_n, -timestamped, -temporally and -chronologically ordered relational sequences of relational data comprising medical and surgical specialties-, diagnosis-, syndrome-, medical history-, categories- and headings-specific present illnesses, past medical history, past surgical history, vaccinations, general health, infectious diseases, sources of infections, accidents, injuries, repetitive strain injuries, hospitalizations, social history, substance abuse, recreation drug use, family history, travel history, occupations, avocations, assessments and plans, allergies, sideeffects and complications, and shared dispensations, prescriptions, distributions, therapeutic and efficacious of pharmaceuticals and medications, supplements, procedures, treatments, therapies, outcomes, comments and feedbacks, and resources and health, healthcare and related life and long-term, financials and insurance services and resources and related and related extrapolations, preemptions, preventions, treatments and acute, subacute, chronic and long-term managements and in any combinations thereof of said DXs (collectively known as related continua in said DXs of said DXs) wherein said relational data, relations of relational data and relational data in said relational data and said relations are associated with said-networked repositories identifications of disseminating, requesting and recipient networked-repositories and said-networked repositories identifications in, between and among individual groups of said networked repositories wherein said relational data, relations of relational data and relational data in said relational data and said relations are associated with said-processor units rendered cumulatives of assigned counts of numbers of said-networked repositories selected, rendered and useages, ranges of numbers of counts from low numbers to high or highest numbers for rendering and dispensing, disseminating and sharing or networked repositories pushing DXs and related continua in, between and among said disseminating, requesting and recipient networked repositories and individual groups of said networked repositories and in, between and among said networked repositories in said individual groups, system of range of numbers of counts for improving and expanding said EDRs and GEDRs and for assisting IL for 24/7 rendering and/or transforming said successive disseminated DXs and related continua thence improved and expand lattices, ECK, EL and IL; and
interactive selectable and clickable interactive DXs and related continua for rendering and successively dispensing, disseminating, managing and sharing or networked repositories pushing DXs and related continua in, between and among said viewing screens including touchscreens for rendering and successively dispensing, disseminating, managing and sharing or networked repositories pushing DXs and related continua in, between and among disseminating, recipient and requesting networked repositories and individual groups of relavant said networked repositories thence improved and expanded lattices, ECK, EL and IL.

6. The system according to claim 1 wherein said seed snippets* comprise integral snippets*, relations of integral snippets*, relations of integral snippets* in, between and among relations of integral snippets*, transient snippets*, relations of transient snippets*, relations of transient snippets* in, between and among relations of transient snippets*, relations of transient snippets* in, between and among relations of integral snippets* and transient snippets* in EDRs and GEDRs in said lattices in ECK; said relational data and relations of relational data in said EDRs and GEDRs in ECK; said words, terms, phrases, graphics, pictures, videos, relational digital contents, phrases, sentences and any combinations thereof in SDC; and relational data and relations of relational data in said SDC; and any combinations thereof in said ECK and SDC.

7. The system according to claim 1 wherein EL for rendering disseminated DXs and related continua, EL being innate in said-networked repositories-rendered extremely large and expanding multi-dimensional electronic and digital lattices of multi-dimensional individual relations of integral snippets* (known herein as EDRs) and relations of, between and among said integral snippets* in, between and among EDRs (known herein as GEDRs) in ECK comprises said electronic EDRs and GEDRs generator for rendering and successively and improving extremely large and expanding multi-dimensional electronic and digital lattices of multi-dimensional individual relations of integral snippets* (known herein as EDRs) and relations of, between and among said integral snippets* in, between and among EDRs (known herein as GEDRs) in ECK in, between and among disseminating, recipient and requesting networked repositories and individual groups of relavant said networked repositories wherein:
i. each integral snippets* comprising relational words, terms, phrases, graphics, pictures, videos, relational digital contents, phrases, sentences and any combinations thereof each of which is associated with:
first, any and any combinations of industries-, specialties-, categories- and headings-specific goods, products, services and resources and related financials and insurance products, services and resources related and pertained to and associated with health, healthcare, homecare, chronic care and pharmaceuticals industries, specialties, categories and headings (herein this claim known as FIRST);
second, said-networked-repositories-rendered identifications of disseminating, requesting and recipient networked-repositories and said-networked-repositories-rendered identifications in individual groups of relevant disseminating, requesting and recipient networked-repositories; associated with said FIRST;
third, associated with said-processor units rendered cumulatives of assigned counts of numbers of said-networked repositories selected, rendered and useages for ranking and ordering relevances and significances for assisting IL IL for 24/7 rendering and/or transforming said successive disseminated DXs and related continua;
ii. each of integral snippets* and each of relations of integral snippets* is associated with: first, any and any combinations of industries-, specialties-, categories- and headings-specific goods, products, services and resources and related financials and insurance products, services and resources related and pertained to and associated with health, healthcare, homecare, chronic care and pharmaceuticals industries, specialties, categories and headings (herein this claim known as FIRST);
second, said-networked-repositories-rendered identifications of disseminating, requesting and recipient networked-repositories and said-networked-repositories-rendered identifications in individual groups of relevant disseminating, requesting and recipient networked-repositories associated with said FIRST); and
third, associated with said-processor units rendered cumulatives of assigned counts of numbers of said-networked repositories selected, rendered and useages for ranking and ordering relevances and significances for assisting IL IL for 24/7 rendering and/or transforming said successive disseminated DXs and related continua.

8. The system according to claim 1 wherein IL for 24/7 rendering and/or transforming said successive disseminated DXs and related continua as individual relations of integral snippets* and relations of, between and among said integral snippets* associating with said-networked repositories identifications of disseminating, requesting and recipient networked-repositories and said-networked repositories identifications in individual groups of said networked repositories into EDRS and GEDRs in ECK thence expanded and improved lattices and EL thence UL thence said disseminations, dispensations and sharing or networked repositories pushing of said disseminated DXs and related continua in, between and among said relevant disseminating, requesting and recipient networked-repositories, IL for:
instructing disseminating, recipient and requesting networked repositories for successively scan and match said successively selected and rendered transient snippets* and relations of transient snippets* in relational databases with same, similar and/or approximate integral snippets* and relations of integral snippets* in EDRs and GEDRs in ECK and, IL for:
Scans and Matches 1—selecting and rendering transient snippets* and/or relations of transient snippets* same as integral snippets* and/or relations of integral snippets* in, between and among relevant EDRs and GEDRs in ECK (known herein as fits fit or seed snippets*), IL for finding the fits of said renderings with same integral snippets* and relations of integral snippets* in ECK, IL for instructing said processor units for counting and associating a range of counts of low numbers to high numbers of incidences and accountings of successful insertions, placements and cumulations of said fits as integral snippets* and/or said relations of same DXs and related continua associating with, hidden, encrypted or deleted said-identifications of disseminating-networked-repositories and recipient networked-repositories into said relational databases wherein IL for instructing said processor units for continually rank said accountings between and among said fits and associating similars, approximates and transformed (plural) in ECK in, between and among networked devices comprising said viewing screens including touchscreens for rendering, dispensing, disseminating, managing and sharing or networked repositories pushing DXs and related continua associating with, hidden, encrypted or deleted said-identifications of disseminating networked-repositories to in recipient networked-repositories in, between and among said networked communication devices wherein said electronic EDRs and GEDRs generator for repeatedly effecting, expanding and improving DXs and related continua thence ECK thence EL among said networked repositories;
Scans and Matches 2a.—if only selected and rendered transient snippets* and/or relations of transient snippets* are similar to integral snippets* and/or relations of integral snippets* in EDRs and GEDRs in ECK (herein known as similars), further to Scans and Matches 1, IL for instructing said processor units for accounting said similars relating to relevant EDRs and GEDRs in ECK, and IL for instructing said processor units for counting and associating a range of counts of low numbers to high numbers of incidences and accountings of similars relative to said accountings of said fits based on DXs and related continua and SDC and, first, said processor units for scanning and matching and applying said selected and rendered transient snippets* and/or relations of transient snippets* to EDRs and GEDRs in ECK, said processor units for finding similars being said fits and similars close to said finds;
second, further to Scans and Matches 1., successively replacing similar selected and rendered transient snippets* and relations of transient snippets* with integral snippets* and relations of integral snippets* in said similars, IL for getting said replacements from integral snippets* and relations of integral snippets* in DXs and related continua based on EL in ECK and SDC;
third, further to Scans and Matches 1. and said second, transforming selected and rendered transient snippets* and/or relations of transient snippets* into integral snippets* and/or relations of integral snippets* in similars, and IL for sequentially editing selected and rendered similar transient snippets* and/or relations of transient snippets* by tweaking the arrangements of relational data in and/or adding, inserting and/or deleting relational data into individual selected and rendered transient snippets* and/or relations of transient snippets* in similars effected by IL for rendering said plurality of transformed (plural) by mirroring said transformed (plural) to integral snippets* and relations of integral snippets* in DXs and related continua in ECK and SDC, and said innovative computer programs instructing said processor units for counting and associating a range of counts of low numbers to high numbers of incidences and accountings of similars relative to said accountings of said fits, similars and said transformed (plural) in said relational databases in ECK thence improved EL;
Scans and Matches 2b.—further to Scans and Matches 1. and 2a., if only selected and rendered transient snippets* and/or relations of transient snippets* approximate integral snippets* and/or relations of integral snippets* in EDRs and GEDRs in ECK (herein known as approximates); fourth, IL for successively replacing approximate selected and rendered transient snippets* and relations of transient snippets* with integral snippets* and relations of integral snippets* in said approximates, said processor units for getting said replacements from DXs and related continua in ECK based on EL and SDC;
fifth, further to Scans and Matches 1a. and said fourth, IL for transforming selected and rendered approximate transient snippets* and/or relations of transient snippets* into integral snippets* and/or relations of integral snippets* in approximates, whereby IL for sequentially editing selected and rendered similar transient snippets* and/or relations of transient snippets* by tweaking the arrangements of relational data in and/or adding, inserting into and/or deleting relational data in individual selected and rendered transient snippets* and/or relations of transient snippets* in approximates effected by IL for rendering said plurality of transformed (plural) by mirroring said transformed (plural) to integral snippets* and relations of integral snippets* in DXs and related continua in ECK and SDC based on EL, and said innovative computer programs instructing said processor units for counting and associating a range of counts of low numbers to high numbers of incidences and accountings of similars relative to said accountings of said fits, similars and said transformed (plural) in said relational databases in ECK;
said fits or seed snippets* and transformed or seed snippets* for instructing processor units for rendering, dispensing, disseminating and sharing or networked repositories pushing relevant DXs and related continua comprising individual relations of relational data comprising industries-, specialties-, categories- and headings-specific goods, products, services and resources and related financials and insurance products, services and resources related and pertained to and associated with health, healthcare, homecare, chronic care and pharmaceuticals industries, specialties, categories and headings, and individual relations of, between and among said relational data in said individual relations of relational data, respectively, said relational data and in said relational data are associated with said-networked repositories identifications of disseminating, requesting and recipient networked-repositories and individual groups of said-networked repositories identifications in, between and among said disseminating, requesting and recipient networked repositories and in, between and among individual groups of relevant networked repositories in EDRS and GEDRs thence expanding and improving lattices thence improved improved ECK thence improved EL and IL and vice versa;
said said-networked repositories identifications of disseminating, requesting and recipient networked-repositories and individual groups of said-networked repositories identifications for inserting said fits and transformed (plural) into EDRs and GEDRS in ECK;
said electronic EDRs and GEDRs generator for repetitively get, retrieve and display said EDRs and GEDRs associating with a range of assigned low to high counts of EDRs and GEDRs comprising fits, similars, approximates and transformed (plural) associating with ranks and probabilities of said displayed EDRs and GEDRs accurately related to disseminating, requesting and recipient networked-repositories rendering said fits, similars, approximates and transformed (plural) in said viewing screens including touchscreen for furthering successive selections and renderings to updating and eventually advancing said selected and rendered as outcomes and products of said Scans and Matches 1 thence improved ECK and EL or rendering redundant similars and approximates by said processor units due to relatively decreasing uses or disuses by, between and among said networked repositories; and
IL for repetitively get and retrieve said EDRs and GEDRs as interactive DXs and related continua comprising ranks and orders of fits, similars, approximates and transformed (plural) in ECK in, between and among said viewing screens including touchscreens for successively effecting either successive enhancements by frequent selections of fits or by extinctions by less frequent selections or disuses of similars and/or approximates thence improved and affirmed EDRs and GEDRs in ECK and EL, as said electronic EDRs and GEDRs generator for repeatedly rendering and dispensing, disseminating, managing and sharing or networked repositories pushing DXs and related continua in, between and among said viewing screens including touchscreens for repeatedly effecting, expanding and improving DXs and related continua thence ECK thence EL in and among said networked repositories.

9. In a system of networked repositories-rendered innate thinking faculty possessing logics for instructing processor units (henceforth known as IL) and expansive cumulative knowledge comprising extremely large and expanding multi-dimensional electronic and digital lattices of multi-dimensional relational databases comprising EDRs and GEDRs in relational databases in memories (henceforth collectively known as ECK) wherein ECK possessing innate expansive logics for instructing said processor units (henceforth known as EL) as consequences of the existence of said lattices in memories and storage means (herein known as memories), said innate thinking faculty comprising the innovative computer programs possessing IL in said memories attaching to processor units in, between and among said networked repositories wherein expanding ECK for improving EL in, between and among disseminated DXs and related continua in, between and among disseminating, recipient and requesting networked repositories wherein EL for improving IL for 24/7 rendering disseminated DXs and related continua in, between and among said disseminating, recipient and requesting networked repositories, a abiotic intelligence-rendered method (henceforth AIR method) for said networked repositories rendering, dispensing, disseminating and sharing or networked repositories pushing individual diagnoses and continua for assisting, improving and optimizing people's health, healthcare, homecare, quality of life, standard of living, longevity and independence and freedom to remain, dwell and live in their homes and dwellings at various and all stages of life and socioeconomics of billions of users at the lowest cost (known herein as DXs and related continua) in, between and among said networked repositories associating with said billions and in said viewing screens including touchscreens as interactive EDRs and GEDRs of selectable and clickable interactive DXs and related continua for rendering and successively improving EDRs and GEDRs in ECK thence improved EL, said improved EL for instructing said processor units for rendering and successively rendering, dispensing, disseminating, managing and sharing or networked repositories pushing DXs and related continua in, between and among networked repositories and viewing screens including touchscreens, said method comprises processes of:
said innovative computer programs instructing said processor units to rendering the electronic EDRs and GEDRs generator to rendering and successively improving Source DXs and related continua comprising extremely large and expanding multi-dimensional electronic and digital lattices of multi-dimensional relational databases comprising EDRs and GEDRs in relational databases (known herein as SDC) in said memories, said electronic EDRs and GEDRs generator to rendering and successively disseminating and sharing or networked repositories pushing said SDC as interactive SDC comprising interactive SDC in, between and among said networked repositories;
said electronic EDRs and GEDRs generator getting and rendering SDC as interactive SDC-rendered interactive EDRs and GEDRs comprising interactive relations of interactive integral snippets* and individual interactive integral snippets* of, between and among said individual relations associating with said-networked-repositories said-networked repositories identifications, said interactives dispensing, disseminating and sharing or networked repositories pushing DXs and related continua between and among said disseminating, requesting and recipient-networked-repositories and in, between and among relevant individual groups of said networked repositories whose said-networked repositories identifications for rendering and dispensing, disseminating and sharing or networked repositories pushing successive disseminated and shared DXs and related continua in, between and among said individual groups of said networked repositories in said networked devices comprising viewing screens including touchscreens for rendering, disseminating and sharing or networked repositories pushing SDC as interactive EDRs and GEDRs comprising relations of chronological and temporal sequences and orders of selectable and clickable interactive DXs and related continua rendering and successively improving and expanding EDRs and GEDRs related to identifications of said networked repositories and in individual groups of networked repositories in, between and among said networked repositories to improving and expanding said EDRs and GEDRs in ECK thence EL in innate thinking faculty thence improved IL;
said electronic EDRs and GEDRs generator rendering and disseminating and sharing or networked repositories pushing seed snippets* to rendering relevant EDRs and GEDRs thence expanded and improved lattices thence ECK thence EL and IL and vice versa in, between and among said networked repositories and for rendering individual groups of said networked repositories;
said seed snippets* instructing processor units to rendering, dispensing, disseminating and sharing or networked repositories or networked repositories pushing relevant EDRs and GEDRs in, between and among said disseminating, requesting and recipient networked repositories and in, between and among networked repositories in individual groups of relevant networked repositories in EDRS and GEDRs thence expanding and improving lattices thence ECK thence EL and IL and vice versa;
said electronic EDRs and GEDRs generator rendering and dispensing, disseminating and sharing or networked repositories pushing individual relations of integral snippets* comprising individual diagnoses including chronic diseases and related diagnoses of diseases, illneses, disorders and sequelae and related continua of current, present and future life and socioeconomic streses, deficiencies, adversities and issues related continua of extrapolations, preemptions, preventions and managements including treatments and solutions (herein collectively known as DXs and related continua) in relational databases (known herein as EDRs, plural) and relations of, between and among said integral snippets* in, between and among EDRs in said relational databases (known herein as GEDRs, plural) in ECK in, between and among disseminating networked repositories and recipient networked repositories wherein electronic EDRs and GEDRs generator for effecting, expanding and improving ECK thence EL in, between and among said networked repositories in networked communication devices comprising viewing screens including touchscreens for rendering, dispensing, disseminating, managing and sharing or networked repositories pushing DXs and related continua associating with said-networked repositories identifications of disseminating, requesting and recipient networked-repositories and said-networked repositories identifications in individual groups of said networked repositories in, between and among said networked communication devices wherein said electronic EDRs and GEDRs generator for rendering and dispensing, disseminating and sharing or networked repositories pushing successive disseminated DXs and related continua comprising selected and rendered individual relations of transient snippets* and/or relations of, between and among transient snippets* in individual of relations transient snippets* associated with said-networked repositories identifications of disseminating, requesting and recipient networked-repositories and individual groups of said-networked repositories identifications for rendering and dispensing, disseminating and sharing or networked repositories pushing successive disseminated DXs and related continua in, between and among relevant individual groups of said networked repositories wherein said innovative computer programs instructing said processor units scanning and matching said successive disseminated DXs and related continua with same, similar and/or approximate integral snippets* and relations of integral snippets* in EDRs and GEDRs in ECK and SDC;
IL 24/7 rendering and/or transforming said successive disseminated DXs and related continua as individual relations of transient snippets* and/or relations of, between and among transient snippets* in individual of relations transient snippets* associating with, hidden, encrypted or deleted said-networked repositories identifications of disseminating, requesting and recipient networked-repositories and for inserting said fits and transformed (plural) into EDRs and GEDRS in ECK;
IL rendering EL to rendering disseminated DXs and related continua comprising relations of integral snippets* and/or relations of integral snippets* associating with, hidden, encrypted or deleted said-networked repositories identifications of disseminating networked-repositories in disseminated EDRs and GEDRs, respectively, into the relational databases in recipient networked-repositories comprising said integral snippets* and/or said relations rendered into, between and among recipient networked-repositories based on EL inherent in EDRs and GEDRs in ECK thence IL and EL for improving and expanding IL and EL and, further to supra, IL rendering EL to rendering disseminated DXs and related continua comprising relations of integral snippets* and/or relations of integral snippets* associating with, hidden, encrypted or deleted said-networked repositories identifications of disseminating networked-repositories in disseminated EDRs and GEDRs, respectively, into the relational databases in recipient networked-repositories comprising said integral snippets* and/or said relations rendered into, between and among recipient networked-repositories based on EL inherent in ECK thence said IL and EL;
said IL and EL to successively transforming similar or approximate said integral snippets* and relations of integral snippets* into integral snippets* and relations of integral snippets* into same integral snippets* and relations of integral snippets* in ECK, said IL and EL to successively deleting, editing instant and successive different selected and rendered DXs and related continua in said successively rendered transient snippets* and relation of transient snippets* and said processor units for successively inserting relational information and data in said integral snippets* and relations of integral snippets* in EDRs and GEDRs in ECK and said processor units successively replacing individual transient snippets* with integral snippets* in ECK based on ECK and said innovative computer programs instructing said processor units to counting and associating numbers of counts of low numbers to high numbers of incidences of successive repair of DXs and related continua in said integral snippets* and relations of integral snippets* as selected and rendered same, similar or approximate integral snippets and relations of integral snippets in ECK thence improved IL and EL in said memories in, between and among networked devices;
said innovative computer programs instructing said processor units to counting and asociating a range of counts of low numbers to high numbers of incidences and accountings of successful insertions, placements and cumulations of said fits as integral snippets* and/or said relations of same DXs and related continua associating with, hidden, encrypted or deleted said-networked repositories identifications of disseminating-networked-repositories and recipient networked-repositories in said relational databases in ECK in said memories in, between and among networked devices, wherein said electronic EDRs and GEDRs generator rendering, disseminating and sharing or networked repositories pushing EDRs and GEDRs as interactive EDRs and GEDRs comprising interactive relations of integral snippets* related to identifications of said networked repositories and in individual groups of networked repositories by said innovative computer programs for instructing said processor units getting said EDRs and GEDRs comprising integral snippets* and relations of integral snippets*, associating with said-processor units rendered numbers of counts of low numbers to high numbers of said incidences and accountings, and rendering same as interactive EDRs and GEDRs comprising a plurality of relations of chronological and temporal sequences and orders of selectable and clickable interactive DXs and related continua in, between and among viewing screens including touchscreens, said-networked repositories temporally and chronologically selecting and rendering transient snippets* and relation of transient snippets* from said selectable and clickable interactive DXs and related continua, said processor units timestamping and processing said renderings associating with said-networked repositories identifications-specific and temporally and chronologically ordered relational sequences of said DXs and related continua in said rendered transient snippets* and relation of transient snippets* into said relational databases in said memories in, between and among said networked repositories wherein said IL and EL to processing said successively rendered transient snippets* and relation of transient snippets* with same, similar and approximate integral snippets* and relations of integral snippets* in ECK thence improved IL and EL thence innate thinking faculty in, between and among said networked repositories to rendering, dispensing, disseminating, managing and sharing or networked repositories pushing relations of continua of relational goods, products, services and resources and related and related financials and insurance products and services related to said renderings and sharing or networked repositories pushing in said relational databases in, between and among said networked repositories wherein said renderings, sharing or networked repositories pushing and continua to assisting, improving and optimizing people's health, healthcare, homecare, quality of life, standard of living, longevity and independence and freedom to remain, dwell and live in their homes and dwellings at various and all stages of life and socioeconomics of billions of users at the lowest cost in, between and among said networked repositories associating with said billions and in said viewing screens including touchscreens as interactive EDRs and GEDRs of selectable and clickable interactive DXs and related continua to rendering and successively improving EDRs and GEDRs in ECK thence improved EL, said improved EL instructing said processor units to rendering and successively rendering, dispensing, disseminating, managing and sharing or networked repositories pushing DXs and related continua in, between and among networked repositories and viewing screens including touchscreens; and
said networked communication devices which are billions of networked servers, smartphones, laptops and desktops comprising said networked repositories including said disseminating networked repositories, recipient networked repositories and requesting networked repositories, said networked repositories comprising said innovative computer programs installed in said memories attached to said processor units, said memories storing said innovative computer programs, software and said networked repositories-rendered relational databases—structured and unstructured—comprising said IL, EL, ECK and innate thinking faculty and said renderings, sharing or networked repositories pushing and continua and related networked dispensations, disseminations and sharing or networked repositories pushing, said networked devices comprising said viewing screens including touchscreens, cameras, microphones, speech recognition software and apps, commercial operating systems for managing said networked servers and devices, the Internet, commercial networking means, security means and software including web browsers; and wherein said “*” connotes “diverse and different” and “large or infinitely large numbers”.

10. The method according to claim 9 wherein said process of said innovative computer programs possessing the professional and technical terminolgies-lay terminologies thesaurus software to render said electronic EDRs and GEDRs generator to render said SDC, EDRs and GEDRs thence ECK and EL and IL, related renderings, sharing or networked repositories pushing and continua and related networked dispensations and sharing or networked repositories pushing in relational professional terminolgies and technical terminolgies-lay terminologies analogies in English and non-English relational data in said memories in, between and among said networked repositories and in said viewing screens including touchscreens to rendering, improving and expanding said SDC, EDRs and GEDRs thence ECK and EL and to rendering and successively dispensing, disseminating, managing and sharing or networked repositories pushing DXs and related continua in, between and among networked repositories and said viewing screens including touchscreens associated with professional people and laypeople.

11. The method according to claim 9 wherein said process of said electronic EDRs and GEDRs generator to rendering and successively improving Source DXs and related continua comprise process of said networked repositories using said electronic EDRs and GEDRs generator to rendering and successively improving individual relations of relational data comprising industries-, specialties-, categories- and headings-specific goods, products, services and resources and related financials and insurance products, services and resources related and pertained to and associated with health, healthcare, homecare, chronic care and pharmaceuticals industries, specialties, categories and headings;
individual relations of, between and among said relational data in said individual relations of relational data, respectively; and
said relational data are associated with said-networked repositories identifications of rendering networked-repositories.

12. The method according to claim 9 wherein said process of EL rendering disseminated DXs and related continua, EL being innate in said-networked repositories-rendered extremely large and expanding multi-dimensional electronic and digital lattices of multi-dimensional individual relations of integral snippets* (known herein as EDRs) and relations of, between and among said integral snippets* in, between and among EDRs (known herein as GEDRs) in ECK, said process comprise said innovative computer programs instructing said processor units to render said electronic EDRs and GEDRs generator to rendering and successively and improving in, between and among disseminating, recipient and requesting networked repositories and individual groups of relavant said networked repositories:
i. each integral snippets* comprising relational words, terms, phrases, graphics, pictures, videos, relational digital contents, phrases and/or sentences and any combinations thereof each of which is associated with first, any and any combinations of industries-, specialties-, categories- and headings-specific goods, products, services and resources and related financials and insurance products, services and resources related and pertained to and associated with health, healthcare, homecare, chronic care and pharmaceuticals industries, specialties, categories and headings (herein this claim known as FIRST);
second, said-networked-repositories-rendered identifications of disseminating, requesting and recipient networked-repositories and said-networked-repositories-rendered identifications in individual groups of relevant disseminating, requesting and recipient networked-repositories associated with said FIRST;
third, associated with said-processor units rendered cumulatives of assigned counts of numbers of said-networked repositories selected, rendered and useages for ranking and ordering relevances and significances for assisting IL IL for 24/7 rendering and/or transforming said successive disseminated DXs and related continua;
ii. each of integral snippets* and each of relations of integral snippets* is associated with first, any and any combinations of industries-, specialties-, categories- and headings-specific goods, products, services and resources and related financials and insurance products, services and resources related and pertained to and associated with health, healthcare, homecare, chronic care and pharmaceuticals industries, specialties, categories and headings (herein this claim known as FIRST);
second, said-networked-repositories-rendered identifications of disseminating, requesting and recipient networked-repositories and said-networked-repositories-rendered identifications in individual groups of relevant disseminating, requesting and recipient networked-repositories associated with said FIRST; and
third, associated with said-processor units rendered cumulatives of assigned counts of numbers of said-networked repositories selected, rendered and useages for ranking and ordering relevances and significances to assisting IL, IL to 24/7 rendering and/or transforming said successive disseminated DXs and related continua.

13. The method according to claim 9 wherein IL to 24/7 rendering and/or transforming said successive disseminated DXs and related continua as individual relations of integral snippets* and relations of, between and among said integral snippets* associating with said-networked repositories identifications of disseminating, requesting and recipient networked-repositories and said-networked repositories identifications in individual groups of said networked repositories into EDRS and GEDRs in ECK thence expanded and improved lattices and EL thence IL thence said disseminations, dispensations and sharing or networked repositories pushing of said disseminated DXs and related continua in, between and among said relevant disseminating, requesting and recipient networked-repositories, said process comprises processes of IL to instruct disseminating, recipient and requesting networked repositories to successively scan and match said successively selected and rendered transient snippets* and relations of transient snippets* in relational databases with same, similars and/or approximate integral snippets* and relations of integral snippets* in EDRs and GEDRs in ECK, and IL to:
Scans and Matches 1—select and render transient snippets* and/or relations of transient snippets* same as integral snippets* and/or relations of integral snippets* in, between and among relevant EDRs and GEDRs in ECK (known herein as fits or seed snippets*), IL to finding the fits of said renderings with same integral snippets* and relations of integral snippets* in ECK, IL to counting and associating a range of counts of low numbers to high numbers of incidences and accountings of successful insertions, placements and cumulations of said fits as integral snippets* and/or said relations of same DXs and related continua associating with, hidden, encrypted or deleted said-identifications of disseminating-networked-repositories and recipient networked-repositories into said relational databases wherein IL to continually rank said accountings between and among said fits and associating similars, approximates and transformed (plural) in ECK in, between and among networked devices comprising said viewing screens including touchscreens for rendering, dispensing, disseminating, managing and sharing or networked repositories pushing DXs and related continua associating with, hidden, encrypted or deleted said-identifications of disseminating networked-repositories to in recipient networked-repositories in, between and among said networked communication devices whereby said electronic EDRs and GEDRs generator repeatedly effecting, expanding and improving DXs and related continua thence ECK thence EL among said networked repositories;
Scans and Matches 2a.—if and/or only selected and rendered transient snippets* and/or relations of transient snippets* are similar to integral snippets* and/or relations of integral snippets* in EDRs and GEDRs in ECK (herein known as similars), further to Scans and Matches 1, IL to accounting said similars relating to relevant EDRs and GEDRs in ECK, and IL to counting and associating a range of counts of low numbers to high numbers of incidences and accountings of similars relative to said accountings of said fits base on SDC and,
first, said processor units scanning and matching and applying said selected and rendered transient snippets* and/or relations of transient snippets* to EDRs and GEDRs in ECK, said processor units for finding similars being said fits and similars close to said finds;
second, further to Scans and Matches 1., IL to successively replacing similar selected and rendered transient snippets* and relations of transient snippets* with integral snippets* and relations of integral snippets* in said similars, IL to getting said replacements from integral snippets* and relations of integral snippets* in DXs and related continua in ecK and SDC; third, further to Scans and Matches 1. and said second, IL to transforming selected and rendered transient snippets* and/or relations of transient snippets* into integral snippets* and/or relations of integral snippets* in similars, whereby L to sequentially editing selected and rendered similar transient snippets* and/or relations of transient snippets* by tweaking the arrangements of relational data in and/or adding, inserting and/or deleting relational data into individual selected and rendered transient snippets* and/or relations of transient snippets* in similars effected effected by IL to render said plurality of transformed (plural) by mirroring said transformed (plural) to integral snippets* and relations of integral snippets* in in DXs and related continua in ECK and SDC, and said innovative computer programs instructing said processor units to counting and associating a range of counts of low numbers to high numbers of incidences and accountings of similars relative to said accountings of said fits, similars and said transformed (plural) in said relational databases in ECK;
Scans and Matches 2b.—further to Scans and Matches 1. and 2a., if and/or selected and rendered transient snippets* and/or relations of transient
snippets* approximate integral snippets* and/or relations of integral snippets* in EDRs and GEDRs in ECK (herein known as approximates);
fourth, further to Scans and Matches 1a., IL to successively replacing approximate selected and rendered transient snippets* and relations of transient snippets* with integral snippets* and relations of integral snippets* in said approximates, said processor units for getting said replacements from DXs and related continua in ECK and SDC;
fifth, further to Scans and Matches 1a. and said fourth, IL to transforming selected and rendered approximate transient snippets* and/or relations of transient snippets* into integral snippets* and/or relations of integral snippets* in approximates, whereby IL to sequentially editing selected and rendered similar transient snippets* and/or relations of transient snippets* by tweaking the arrangements of relational data in and/or adding, inserting into and/or deleting relational data in individual selected and rendered transient snippets* and/or relations of transient snippets* in approximates effected by IL to render said plurality of transformed (plural) by mirroring said transformed (plural) to integral snippets* and relations of integral snippets* in in DXs and related continua in ECK and SDC, and said innovative computer programs instructing said processor units to counting and associating a range of counts of low numbers to high numbers of incidences and accountings of similars relative to said accountings of said fits, similars and said transformed (plural) in said relational databases in ECK;
said fits or seed snippets* and transformed or seed snippets* instructing processor units to rendering, dispensing, disseminating and sharing or networked repositories pushing relevant DXs and related continua comprising individual relations of relational data comprising industries-, specialties-, categories- and headings-specific goods, products, services and resources and related financials and insurance products, services and resources related and pertained to and associated with health, healthcare, homecare, chronic care and pharmaceuticals industries, specialties, categories and headings, and individual relations of, between and among said relational data in said individual relations of relational data, respectively, said relational data and in said relational data are associated with said-networked repositories identifications of disseminating, requesting and recipient networked-repositories and individual groups of said-networked repositories identifications in, between and among said disseminating, requesting and recipient networked repositories and in, between and among individual groups of relevant networked repositories in EDRS and GEDRs thence expanding and improving lattices thence improved ECK thence improved EL and IL and vice versa;
said electronic EDRs and GEDRs generator to get, retrieve and display said EDRs and GEDRs associating with a range of assigned low to high counts of EDRs and GEDRs comprising fits, similars, approximates and transformed (plural) associating with ranks and probabilities of said displayed EDRs and GEDRs accurately related to disseminating networked-repositories and recipient networked-repositories rendering said fits, similars, approximates and transformed (plural) in said viewing screens including touchscreen for furthering successive selections and renderings to updating and eventually advancing said selected and rendered as outcomes and products of said Scans and Matches 1 or rendering redundant similars and approximates by said processor units due to relatively decreasing uses or disuses by, between and among said networked repositories; and
IL to repetitively get and retrieve said EDRs and GEDRs as interactive DXs and related continua comprising ranks and orders of fits, similars, approximates and transformed (plural) in ECK in, between and among said viewing screens including touchscreens for successively effecting either successive enhancements by frequent selections of fits or by extinctions by less frequent selections or disuses of similars and/or approximates thence improved and affirmed EDRs and GEDRs in ECK, IL and EL, as said electronic EDRs and GEDRs generator repeatedly rendering and dispensing, disseminating, managing and sharing or networked repositories pushing DXs and related continua in, between and among said viewing screens including touchscreens for repeatedly effecting, expanding and improving DXs and related continua thence ECK thence EL in, between and among said networked repositories.

14. In a system of networked repositories-rendered innate thinking faculty possessing logics for instructing processor units (henceforth known as IL) and expansive cumulative knowledge comprising extremely large and expanding multi-dimensional electronic and digital lattices of multi-dimensional relational databases comprising EDRs and GEDRs in relational databases in memories (henceforth collectively known as ECK) wherein ECK possessing innate expansive logics for instructing said processor units (henceforth known as EL) as consequences of the existence of said lattices in memories and storage means (herein known as memories), said innate thinking faculty comprising the innovative computer programs possessing IL in said memories attaching to processor units in, between and among said networked repositories wherein expanding ECK for improving EL in, between and among disseminated DXs and related continua in, between and among disseminating, recipient and requesting networked repositories and individual groups of said-networked repositories identifications of said networked repositories for inserting said fits and transformed (plural) into EDRs and GEDRS in ECK wherein EL for improving IL for 24/7 rendering disseminated and shared DXs and related continua in, between and among said disseminating, recipient, requesting networked repositories and individual groups of relevant networked repositories whose networked-repositories identifications are in and associating relational data in and associating with integral snippets* and relations of integral snippets in EDRs and GEDRs in ECK, a method of said innovative computer programs rendering the electronic EDRs and GEDRs generator to 24/7 rendering and dispensing, disseminating and sharing or networked repositories pushing DXs and related continua in, between and among said disseminating, recipient, requesting networked repositories and said individual groups whereby IL and EL 24/7 rendering and/or transforming disseminated DXs and related continua comprising individual relations of transient snippets* and/or relations of, between and among transient snippets* in individual of relations transient snippets* associated with said-networked repositories identifications of disseminating, requesting and recipient networked-repositories and said individual groups as individual relations of integral snippets* and/or relations of, between and among integral snippets* in individual of relations integral snippets* associating with said-networked repositories identifications in EDRs and GEDRS in ECK thence enhanced and empowered EL to more accurately improving rendering and/or transforming successive disseminated DXs and related continua in, between and among said networked repositories and said individual groups, and consequently, IL is more efficiently or optimized to rendering EDRs and GEDRS in ECK thence consequently improved and empowered EL from successive disseminated DXs and related continua to assisting, improving and optimizing people's health, healthcare, homecare, quality of life, standard of living, longevity and independence and freedom to remain, dwell and live in their homes and dwellings at various and all stages of life and socioeconomics of billions of users at the lowest cost in, between and among said networked repositories associating with said billions and in said viewing screens including touchscreens, said method comprises processes of:
said innovative computer programs instructing said processor units to rendering the electronic EDRs and GEDRs generator to rendering and successively improving Source DXs and related continua comprising extremely large and expanding multi-dimensional electronic and digital lattices of multi-dimensional relational databases comprising EDRs and GEDRs in relational databases (known herein as SDC) in said memories, said electronic EDRs and GEDRs generator to rendering and successively disseminating and sharing or networked repositories pushing said SDC as interactive SDC comprising interactive SDC in, between and among said networked repositories;
said electronic EDRs and GEDRs generator getting and rendering SDC as interactive SDC-rendered interactive EDRs and GEDRs comprising interactive relations of interactive integral snippets* and individual interactive integral snippets* of, between and among said individual relations associating with said-networked-repositories said-networked repositories identifications, said interactives dispensing, disseminating and sharing or networked repositories pushing DXs and related continua between and among said disseminating, requesting and recipient-networked-repositories and in, between and among relevant individual groups of said networked repositories whose said-networked repositories identifications for rendering and dispensing, disseminating and sharing or networked repositories pushing successive disseminated and shared DXs and related continua in, between and among said individual groups of said networked repositories in said networked devices comprising viewing screens including touchscreens for rendering, disseminating and sharing or networked repositories pushing SDC as interactive EDRs and GEDRs comprising relations of chronological and temporal sequences and orders of selectable and clickable interactive DXs and related continua rendering and successively improving and expanding EDRs and GEDRs related to identifications of said networked repositories and in individual groups of networked repositories in, between and among said networked repositories to improving and expanding said EDRs and GEDRs in ECK thence EL in innate thinking faculty thence improved IL;
said electronic EDRs and GEDRs generator rendering and disseminating and sharing or networked repositories pushing seed snippets* to rendering relevant EDRs and GEDRs thence expanded and improved lattices thence ECK thence EL and IL and vice versa in, between and among said networked repositories and for rendering individual groups of said networked repositories;
said seed snippets* instructing instructing processor units to rendering, dispensing, disseminating and sharing or networked repositories or networked repositories pushing relevant EDRs and GEDRs in, between and among said disseminating, requesting and recipient networked repositories and in, between and among networked repositories in individual groups of relevant networked repositories in EDRS and GEDRs thence expanding and improving lattices thence ECK thence EL and IL and vice versa;
said electronic EDRs and GEDRs generator rendering and dispensing, disseminating and sharing or networked repositories pushing dispensing, disseminating and sharing or networked repositories pushing individual relations of integral snippets' comprising individual diagnoses including chronic diseases and related diagnoses of diseases, illneses, disorders and sequelae and related continua of current, present and future life and socioeconomic streses, deficiencies, adversities and issues related continua of extrapolations, preemptions, preventions and managements including treatments and solutions (herein collectively known as DXs and related continua) in relational databases (known herein as EDRs, plural) and relations of, between and among said integral snippets* in, between and among EDRs in said relational databases (known herein as GEDRs, plural) in ECK in, between and among disseminating networked repositories and recipient networked repositories wherein electronic EDRs and GEDRs generator for effecting, expanding and improving ECK thence EL in, between and among said networked repositories in networked communication devices comprising viewing screens including touchscreens for rendering, dispensing, disseminating, managing and sharing or networked repositories pushing DXs and related continua associating with said-networked repositories identifications of disseminating, requesting and recipient networked-repositories and said-networked repositories identifications in individual groups of said networked repositories in, between and among said networked communication devices wherein said electronic EDRs and GEDRs generator for rendering and dispensing, disseminating and sharing or networked repositories pushing successive disseminated DXs and related continua comprising selected and rendered individual relations of transient snippets* and/or relations of, between and among transient snippets* in individual of relations transient snippets* associated with said-networked repositories identifications of disseminating, requesting and recipient networked-repositories and individual groups of said-networked repositories identifications for rendering and dispensing, disseminating and sharing or networked repositories pushing successive disseminated DXs and related continua in, between and among relevant individual groups of said networked repositories wherein said innovative computer programs instructing said processor units scanning and matching said successive disseminated DXs and related continua with same, similar and/or approximate integral snippets* and relations of integral snippets* in EDRs and GEDRs in ECK and SDC;
IL 24/7 rendering and/or transforming said successive disseminated DXs and related continua as individual relations of transient snippets* and/or relations of, between and among transient snippets* in individual of relations transient snippets* associating with, hidden, encrypted or deleted said-networked repositories identifications of disseminating, requesting and recipient networked-repositories and for inserting said fits and transformed (plural) into EDRs and GEDRS in ECK;
IL rendering EL to rendering disseminated DXs and related continua comprising relations of integral snippets* and/or relations of integral snippets* associating with, hidden, encrypted or deleted said-networked repositories identifications of disseminating networked-repositories in disseminated EDRs and GEDRs, respectively, into the relational databases in recipient networked-repositories comprising said integral snippets* and/or said relations rendered into, between and among recipient networked-repositories based on EL inherent in EDRs and GEDRs in ECK thence IL and EL for improving and expanding IL and EL and, further to supra, IL rendering EL to rendering disseminated DXs and related continua comprising relations of integral snippets* and/or relations of integral snippets* associating with, hidden, encrypted or deleted said-networked repositories identifications of disseminating networked-repositories in disseminated EDRs and GEDRs, respectively, into the relational databases in recipient networked-repositories comprising said integral snippets* and/or said relations rendered into, between and among recipient networked-repositories based on EL inherent in ECK thence said IL and EL;
said IL and EL to successively transforming similar or approximate said integral snippets* and relations of integral snippets* into integral snippets* and relations of integral snippets* into same integral snippets* and relations of integral snippets* in ECK, said IL and EL to successively deleting, editing instant and successive different selected and rendered DXs and related continua in said successively rendered transient snippets* and relation of transient snippets* and said processor units for successively inserting relational information and data in said integral snippets* and relations of integral snippets* in EDRs and GEDRs and said processor units successively replacing individual transient snippets* with integral snippets* in ECK based on ECK and said innovative computer programs instructing said processor units to counting and associating numbers of counts of low numbers to high numbers of incidences of successive repair of DXs and related continua in said integral snippets* and relations of integral snippets* as selected and rendered same, similar or approximate integral snippets and relations of integral snippets in ECK thence improved IL and EL in said memories in, between and among networked devices;
said innovative computer programs instructing said processor units to counting and associating a range of counts of low numbers to high numbers of incidences and accountings of successful insertions, placements and cumulations of said fits as integral snippets* and/or said relations of same DXs and related continua associating with, hidden, encrypted or deleted said-networked repositories identifications of disseminating-networked-repositories and recipient networked-repositories in said relational databases in ECK in said memories in, between and among networked devices, wherein said electronic EDRs and GEDRs generator rendering, disseminating and sharing or networked repositories pushing EDRs and GEDRs as interactive EDRs and GEDRs comprising interactive relations of integral snippets* related to identifications of said networked repositories and in individual groups of networked repositories by said innovative computer programs for instructing said processor units getting said EDRs and GEDRs comprising integral snippets* and relations of integral snippets*, associating with said-processor units rendered numbers of counts of low numbers to high numbers of said incidences and accountings, and rendering same as interactive EDRs and GEDRs comprising a plurality of relations of chronological and temporal sequences and orders of selectable and clickable interactive DXs and related continua in, between and among viewing screens including touchscreens, said-networked repositories temporally and chronologically selecting and rendering transient snippets* and relation of transient snippets* from said selectable and clickable interactive DXs and related continua, said processor units timestamping and processing said renderings associating with said-networked repositories identifications-specific and temporally and chronologically ordered relational sequences of said DXs and related continua in said rendered transient snippets* and relation of transient snippets* into said relational databases in said memories in, between and among said networked repositories wherein said IL and EL to process said successively rendered transient snippets* and relation of transient snippets* with same, similar and approximate integral snippets* and relations of integral snippets* in ECK thence improved IL and EL thence innate thinking faculty in, between and among said networked repositories to rendering, dispensing, disseminating, managing and sharing or networked repositories pushing relations of continua of relational goods, products, services and resources and related and related financials and insurance products and services related to said renderings and sharing or networked repositories pushing in said relational databases in, between and among said networked repositories wherein said renderings, sharing or networked repositories pushing and continua to assisting, improving and optimizing people's health, healthcare, homecare, quality of life, standard of living, longevity and independence and freedom to remain, dwell and live in their homes and dwellings at various and all stages of life and socioeconomics of billions of users at the lowest cost in, between and among said networked repositories associating with said billions and in said viewing screens including touchscreens as interactive EDRs and GEDRs of selectable and clickable interactive DXs and related continua to rendering and successively improving EDRs and GEDRs in ECK thence improved EL, said improved EL instructing said processor units to rendering and successively rendering, dispensing, disseminating, managing and sharing or networked repositories pushing DXs and related continua in, between and among networked repositories and viewing screens including touchscreens;
said networked communication devices which are billions of networked servers, smartphones, laptops and desktops comprising said networked repositories including said disseminating networked repositories, recipient networked repositories and requesting networked repositories, said networked repositories comprising said innovative computer programs installed in said memories attached to said processor units, said memories storing said innovative computer programs, software and said networked repositories-rendered relational databases—structured and unstructured—comprising said IL, EL, ECK and innate thinking faculty and said renderings, sharing or networked repositories pushing and continua and related networked dispensations, disseminations and sharing or networked repositories pushing, said networked devices comprising said viewing screens including touchscreens, cameras, microphones, speech recognition software and apps, commercial operating systems for managing said networked servers and devices, the Internet, commercial networking means, security means and software
including web browsers; and
wherein said “*” connotes “diverse and different” and “large or infinitely large numbers”.

15. The method according to claim 14 wherein said process of said innovative computer programs possessing the professional and technical terminolgies-lay terminologies thesaurus software to render said electronic EDRs and GEDRs generator to render said SDC, EDRs and GEDRs thence ECK and EL and IL, related renderings, sharing or networked repositories pushing and continua and related networked dispensations and sharing or networked repositories pushing in relational professional terminolgies and technical terminolgies-lay terminologies analogies in English and non-English relational data in said memories in, between and among said networked repositories and in said viewing screens including touchscreens to rendering, improving and expanding said SDC, EDRs and GEDRs thence ECK and EL and to rendering and successively dispensing, disseminating, managing and sharing or networked repositories pushing DXs and related continua in, between and among networked repositories and said viewing screens including touchscreens associated with professional people and laypeople.

16. The method according to claim 14 wherein said process of said electronic EDRs and GEDRs generator to rendering and successively improving Source DXs and related continua comprise process of said networked repositories using said electronic EDRs and GEDRs generator to rendering and successively improving individual relations of relational data comprising industries-, specialties-, categories- and headings-specific goods, products, services and resources and related financials and insurance products, services and resources related and pertained to and associated with health, healthcare, homecare, chronic care and pharmaceuticals industries, specialties, categories and headings;
individual relations of, between and among said relational data in said individual relations of relational data, respectively;
said relational data are associated with said-networked repositories identifications of rendering networked-repositories; and wherein said “*” connotes “diverse and different” and “large or infinitely large numbers”.

17. The method according to claim 14 wherein said process of EL rendering disseminated DXs and related continua, EL being innate in said-networked repositories-rendered extremely large and expanding multi-dimensional electronic and digital lattices of multi-dimensional individual relations of integral snippets* (known herein as EDRs) and relations of, between and among said integral snippets* in, between and among EDRs (known herein as GEDRs) in ECK, said process comprise said innovative computer programs instructing said processor units to render said electronic EDRs and GEDRs generator to rendering and successively and improving in, between and among disseminating, recipient and requesting networked repositories and individual groups of relevant said networked repositories:
i. each integral snippets* comprising relational words, terms, phrases, graphics, pictures, videos, relational digital contents, phrases, sentences and any combinations thereof each of which is associated with first, any and any combinations of industries-, specialties-, categories- and headings-specific goods, products, services and resources and related financials and insurance products, services and resources related and pertained to and associated with health, healthcare, homecare, chronic care and pharmaceuticals industries, specialties, categories and headings (herein this claim known as FIRST);
second, said-networked-repositories-rendered identifications of disseminating, requesting and recipient networked-repositories and said-networked-repositories-rendered identifications in individual groups of relevant disseminating, requesting and recipient networked-repositories associated with said FIRST;
third, associated with said-processor units rendered cumulatives of assigned counts of numbers of said-networked repositories selected, rendered and useages for ranking and ordering relevances and significances for assisting IL IL for 24/7 rendering and/or transforming said successive disseminated DXs and related continua;
ii. each of integral snippets* and each of relations of integral snippets* is associated with first, any and any combinations of industries-, specialties-, categories- and headings-specific goods, products, services and resources and related financials and insurance products, services and resources related and pertained to and associated with health, healthcare, homecare, chronic care and pharmaceuticals industries, specialties, categories and headings (herein this claim known as FIRST);
second, said-networked-repositories-rendered identifications of disseminating, requesting and recipient networked-repositories and said-networked-repositories-rendered identifications in individual groups of relevant disseminating, requesting and recipient networked-repositories associated with said FIRST; and
third, associated with said-processor units rendered cumulatives of assigned counts of numbers of said-networked repositories selected, rendered and useages for ranking and ordering relevances and significances to assisting IL, IL to 24/7 rendering and/or transforming said successive disseminated DXs and related continua.

18. The method according to claim 14 wherein IL to 24/7 rendering and/or transforming said successive disseminated DXs and related continua as individual relations of integral snippets* and relations of, between and among said integral snippets* associating with said-networked repositories identifications of disseminating, requesting and recipient networked-repositories and said-networked repositories identifications in individual groups of said networked repositories into EDRS and GEDRs in ECK thence expanded and improved lattices and EL thence IL thence said disseminations, dispensations and sharing or networked repositories pushing of said disseminated DXs and related continua in, between and among said relevant disseminating, requesting and recipient networked-repositories, said process comprises processes of IL to:
instruct disseminating, recipient and requesting networked repositories to successively scan and match said successively selected and rendered transient snippets* and relations of transient snippets* in relational databases with same, similar and/or approximate integral snippets* and relations of integral snippets* in EDRs and GEDRs in ECK, and IL to:
Scans and Matches 1—select and render transient snippets* and/or relations of transient snippets* same as integral snippets* and/or relations of integral snippets* in, between and among relevant EDRs and GEDRs in ECK (known herein as fits or seed snippets*), IL to finding the fits of said renderings with same integral snippets* and relations of integral snippets* in ECK, IL to counting and associating a range of counts of low numbers to high numbers of incidences and accountings of successful insertions, placements and cumulations of said fits as integral snippets* and/or said relations of same DXs and related continua associating with, hidden, encrypted or deleted said-identifications of disseminating-networked-repositories and recipient networked-repositories into said relational databases wherein IL to continually rank said accountings between and among said fits and associating similars, approximates and transformed (plural) in ECK in, between and among networked devices comprising said viewing screens including touchscreens for rendering, dispensing, disseminating, managing and sharing or networked repositories pushing DXs and related continua associating with, hidden, encrypted or deleted said-identifications of disseminating networked-repositories to in recipient networked-repositories in, between and among said networked communication devices whereby said electronic EDRs and GEDRs generator repeatedly effecting, expanding and improving DXs and related continua thence ECK thence EL among said networked repositories;
Scans and Matches 2a.—if and/or only selected and rendered transient snippets* and/or relations of transient snippets* are similar to integral snippets* and/or relations of integral snippets* in EDRs and GEDRs in ECK (herein known as similars), further to Scans and Matches 1, IL to accounting said similars relating to relevant EDRs and GEDRs in ECK, and IL to counting and associating a range of counts of low numbers to high numbers of incidences and accountings of similars relative to said accountings of said fits base on SDC and, first, said processor units scannig and matching and applying said selected and rendered transient snippets* and/or relations of transient snippets* to EDRs and GEDRs in ECK, said processor units for finding similars being said fits and similars close to said finds;
second, further to Scans and Matches 1., IL to successively replacing similar selected and rendered transient snippets* and relations of transient snippets* with integral snippets* and relations of integral snippets* in said similars, IL to getting said replacements from integral snippets* and relations of integral snippets* in DXs and related continua in ecK and SDC; third, further to Scans and Matches 1. and said second, IL to transforming selected and rendered transient snippets* and/or relations of transient snippets* into integral snippets* and/or relations of integral snippets* in similars, whereby IL to sequentially editing selected and rendered similar transient snippets* and/or relations of transient snippets* by tweaking the arrangements of relational data in and/or adding, inserting and/or deleting relational data into individual selected and rendered transient snippets* and/or relations of transient snippets* in similars effected effected by IL to render said plurality of transformed (plural) by mirroring said transformed (plural) to integral snippets* and relations of integral snippets* in in DXs and related continua in ECK and SDC, and said innovative computer programs instructing said processor units to counting and associating a range of counts of low numbers to high numbers of incidences and accountings of similars relative to said accountings of said fits, similars and said transformed (plural) in said relational databases in ECK;
Scans and Matches 2b.—further to Scans and Matches 1. and 2a., if and/or selected and rendered transient snippets* and/or relations of transient snippets* approximate integral snippets* and/or relations of integral snippets* in EDRs and GEDRs in ECK (herein known as approximates); fourth, further to Scans and Matches 1a., IL to successively replacing approximate selected and rendered transient snippets* and relations of transient snippets* with integral snippets* and relations of integral snippets* in said approximates, said processor units for getting said replacements from DXs and related continua in ECK and SDC;
fifth, further to Scans and Matches 1a. and said fourth, IL to transforming selected and rendered approximate transient snippets* and/or relations of transient snippets* into integral snippets* and/or relations of integral snippets* in approximates, whereby IL to sequentially editing selected and rendered similar transient snippets* and/or relations of transient snippets* by tweaking the arrangements of relational data in and/or adding, inserting into and/or deleting relational data in individual selected and rendered transient snippets* and/or relations of transient snippets* in approximates effected by IL to render said plurality of transformed (plural) by mirroring said transformed (plural) to integral snippets* and relations of integral snippets* in in DXs and related continua in ECK and SDC, and said innovative computer programs instructing said processor units to counting and associating a range of counts of low numbers to high numbers of incidences and accountings of similars relative to said accountings of said fits, similars and said transformed (plural) in said relational databases in ECK;
said fits or seed snippets* and transformed or seed snippets* instructing processor units to rendering, dispensing, disseminating and sharing or networked repositories pushing relevant DXs and related continua comprising individual relations of relational data comprising industries-, specialties-, categories- and headings-specific goods, products, services and resources and related financials and insurance products, services and resources related and pertained to and associated with health, healthcare, homecare, chronic care and pharmaceuticals industries, specialties, categories and headings, and individual relations of, between and among said relational data in said individual relations of relational data, respectively, said relational data and in said relational data are associated with said-networked repositories identifications of disseminating, requesting and recipient networked-repositories and individual groups of said-networked repositories identifications in, between and among said disseminating, requesting and recipient networked repositories and in, between and among individual groups of relevant networked repositories in EDRS and GEDRs thence expanding and improving lattices thence improved ECK thence improved EL and IL and vice versa;
said electronic EDRs and GEDRs generator to get, retrieve and display said EDRs and GEDRs associating with a range of assigned low to high counts of EDRs and GEDRs comprising fits, similars, approximates and transformed (plural) associating with ranks and probabilities of said displayed EDRs and GEDRs accurately related to disseminating networked-repositories and recipient networked-repositories rendering said fits, similars, approximates and transformed (plural) in said viewing screens including touchscreen for furthering successive selections and renderings to updating and eventually advancing said selected and rendered as outcomes and products of said Scans and Matches 1 or rendering redundant similars and approximates by said processor units due to relatively decreasing uses or disuses by, between and among said networked repositories; and
IL to repetitively get and retrieve said EDRs and GEDRs as interactive DXs and related continua comprising ranks and orders of fits, similars, approximates and transformed (plural) in ECK in, between and among said viewing screens including touchscreens for successively effecting either successive enhancements by frequent selections of fits or by extinctions by less frequent selections or disuses of similars and/or approximates thence improved and affirmed EDRs and GEDRs in ECK, IL and EL, as said electronic EDRs and GEDRs generator repeatedly rendering and dispensing, disseminating, managing and sharing or networked repositories pushing DXs and related continua in, between and among said viewing screens including touchscreens for repeatedly effecting, expanding and improving DXs and related continua thence ECK thence EL in, between and among said networked repositories.
